(19) United States
(12) Patent Application Publication
(10) Pub. No.: US 2003/0129709 A1
(43) Pub. Date: Jul. 10, 2003

(54) Method for site-directed mutagenesis of nucleic acid molecules using a single primer

(76) Inventors: Olga Makarova, Ann Arbor, MI (US); Emmanuel Kamberov, Ann Arbor, MI (US)

Correspondence Address: MCLEOD MOYNE & REILLY, P.C., 2190 COMMONS PARKWAY, OKEMOS, MI, 48864, US

(21) Appl. No.: 10/286,896

(22) Filed: Nov. 1, 2002

Related U.S. Application Data
(60) Provisional application No. 60/335,345, filed on Nov. 2, 2001.

(51) Int. Cl. C12P 19/34; C12N 15/74; C12N 15/70; C12N 1/21

(52) U.S. Cl. 435/91.2; 435/471; 435/252.3; 435/252.33; 435/320.1

(57) ABSTRACT

Described is an improved method for introducing a site-directed mutation into a DNA sequence of interest in a target DNA using a single oligonucleotide mutagenic primer which is complementary to the DNA sequence to be mutated in the target DNA and wherein the mutation to be introduced into the DNA sequence by the primer is located within the region of complementarity. In a single reaction, the primer is annealed to the target DNA and extended in an extension/polymerization reaction to produce copies of the target DNA comprising the primer. A selection means is used to remove the target DNA and the copies of the target DNA comprising the primer are propagated in host cells. The method can be adapted to include a plurality of primers in the reaction which enables libraries of mutated DNAs to be produced. Also provided are kits for the improved site-directed mutagenesis method.

### CROSS-REFERENCE TO RELATED APPLICATION

This application claims priority to Provisional Application Serial No. 60/335,345 filed Nov. 2, 2001.

### STATEMENT REGARDING FEDERALLY SPONSORED RESEARCH OR DEVELOPMENT

Not applicable.

Reference to a “Computer Listing Appendix submitted on a Compact Disc”

Not Applicable.

### BACKGROUND OF THE INVENTION

(1) Field of the Invention

The present invention relates to an improved method for introducing a site-directed mutation into a DNA sequence of interest in a target DNA using a single oligonucleotide mutagenic primer which is complementary to the DNA sequence to be mutated in the target DNA and wherein the mutation to be introduced into the DNA sequence by the primer is located within the region of complementarity. In a single reaction, the primer is annealed to the target DNA and extended in an extension/polymerization reaction to produce copies of the target DNA comprising the primer. A selection means is used to remove the target DNA and the copies of the target DNA comprising the primer are propagated in host cells. The method can be adapted to include a plurality of primers in the reaction which enables libraries of mutated DNAs to be produced. Also provided are kits for the improved site-directed mutagenesis method.

(2) Description of Related Art

Site-directed mutagenesis, also known as oligonucleotide-directed mutagenesis, has become an important and powerful method for studying protein structure-function relationships, gene expression, and vector modification (For a review, Ling and Robinson, Analyt. Biochem. 254: 157-178 (1997)). All site directed mutagenesis methods are based on the same concept-an oligonucleotide encoding the desired mutation is annealed to one strand of a DNA of interest and serves as a primer for initiation of DNA synthesis. In this manner, the oligonucleotide primer is incorporated into the newly synthesized strand.

Many of the early methods for site-directed mutagenesis required subcloning a DNA of interest which was usually double-stranded into a vector such as M13 to produce a single-stranded DNA as the template. This was because at the time that was the only reliable means for separating the complementary strands to prevent their reannealing during the mutagenesis reaction. These early methods are exemplified by the method of Kunkel, first described in Proc. Natl. Acad. Sci. USA 82: 488-492 (1985) and described in further detail in Sambrook et al. (Molecular Cloning: A Laboratory Manual, Second Edition, Cold Spring, Cold Spring Harbor, N.Y. (1989)). A variation of the Kunkel method which uses to oligonucleotide primers in the mutagenesis reaction is disclosed in U.S. Pat. No. 5,071,743 to Slilaty et al. These early methods were laborious and because they relied upon subcloning the DNA interest into a single-stranded vector, the length of the DNA of interest which could be subcloned was limited.

With the advent of PCR, several new methods for site-directed mutagenesis have been developed (Fukuoka et al., Biochem. Biophys. Res. Commun. 263: 357-360 (1999); Kim and Maas, BioTech. 28: 196-198 (2000); Parikh and Guengerich, BioTech. 24:4 28-431 (1998); Ray and Nickoloff, BioTech. 13: 342-346 (1992); Wang et al., BioTech. 19: 556-559 (1995); Wang and Malcolm, BioTech. 26: 680-682 (1999); Xu and Gong, BioTech. 26: 639-641 (1999)). These new PCR-based methods involved two-step PCR followed by subcloning, which is time consuming and error prone.

More recently, a method was developed which eliminated the need to subclone the amplified DNA fragment. This method is disclosed in U.S. Pat. Nos. 5,789,166 and 5,932,419, both to Bauer et al., and its PCT equivalent, WO97/20950. The method uses two partially complementary mutagenic oligonucleotide primers which are complementary to opposite strands of a circular double-stranded target DNA containing a sequence of interest. In a cyclic linear amplification reaction, the circular double-stranded target DNA is denatured and the primers annealed to their respective strands. A DNA polymerase is used to extend both primers to make mutated complementary copies of their respective strands. The mutated copies are annealed to form a double-stranded mutated copy of the target DNA and the target DNA removed using a selection enzyme which preferentially digests the target DNA. The remaining DNA is transformed into competent host cells.

More recently, a variation of the Bauer et al. method was described by Hogrefe in Strategies 14.3: 74-75 (2001). The method enables one to five mutations to be introduced into a circular double-stranded target DNA which has been methylated using primers which are each complementary to the same strand of the target DNA. After amplification as described in Bauer et al., the 5′ and 3′ ends of the mutated copies are each ligated together in vitro to form single-stranded circular DNA molecules. After treatment with an enzyme which digests methylated and hemimethylated DNA, the single-stranded DNA molecules are propagated into ultracompetent Escherichia coli cells.

Another method for performing site-directed mutagenesis is disclosed in U.S. Pat. No. 5,702,931 to Andrews et al. The method uses a mutagenic oligonucleotide primer to introduce a desired mutation in one site of a double-stranded target DNA and a unique restriction endonuclease site at another site of the target DNA. After rendering the double-stranded target DNA single-stranded, the primer is annealed to its complementary strand, the primer is extended to produce a double-stranded hybrid DNA molecule with a mismatch at the mutation site and the restriction enzyme site. The hybrid DNA is transformed into competent host cells and the mutated DNA screened by restriction analysis.

U.S. Pat. No. 5,780,270 to Lesley and its corresponding PCT application, WO 98/02537, discloses a site-directed mutagenesis method which uses two oligonucleotide primers, one primer to insert a desired mutation into a DNA sequence of interest in a target DNA and the other primer to insert an inactivating mutation in an antibiotic resistance gene comprising the target DNA. After annealing the primers to the target DNA and extending the primers to generate a hybrid DNA, the hybrid DNA is transformed into competent host cells and host cells containing the mutated DNA identified by differential antibiotic resistance screening.

U.S. Pat. No. 6,242,222 B1 to Gifford provides a method for programmed sequential mutagenesis of a target DNA. The method produces a series of mutated DNAs in a programmed sequence over multiple rounds of DNA synthesis. The methods uses a series of mutagenic primers of which some are incapable of binding and promoting polymerization from the target DNA but do bind and promote polymerization from the mutagenized products produced in earlier rounds of the program of sequential mutagenesis.

U.S. Pat. No. 6,136,601 to Meyer, Jr. et al. discloses a method for targeted mutagenesis in living cells using modified oligonucleotides and EPO Application No. EP1178109 A1 disclose a multiple primer method for performing site-directed mutagenesis.

A plurality of modified PCR-based site-directed mutagenesis methods have been developed over the last decade. The following represent some of these methods: Angag and Schutz, Biotech. 30: 486-488 (2001), Wang and Wilkinson, Biotech. 29: 976-978 (2000), Kang et al., Biotech.20: 44-46 (1996), Ogel and McPherson, Protein Engineer. 5: 467-468 (1992), Kirsch and Joly, Nuc. Acids. Res. 26: 1848-1850 (1998), Rhem and Hancock, J. Bacteriol. 178: 3346-3349 (1996), Boles and Miogsa, Curr. Genet. 28: 197-198 (1995), Barrenttino et al., Nuc. Acids. Res. 22: 541-542 (1993), Tessier and Thomas, Meths. Molec. Biol. 57: 229-237, and Pons et al., Meth. Molec. Biol. 67: 209-218.

While the above site-directed mutagenesis methods have proven to be useful for making many short mutations in a target DNA, they are for the most part either inefficient for making, or unable to make, large insertions, deletions, or substitutions in a target DNA. In addition some of the site-directed mutagenesis methods are either cumbersome to perform or require specialized cloning vectors, competent host cells, or the like. Therefore, a need remains for improved methods for performing site-directed mutagenesis which are simple to use, inexpensive, do not require specialized cloning vectors, host cells, and the like, and which enable large insertions, deletions, or substitutions to be made in a target DNA.

### SUMMARY OF THE INVENTION

The present invention relates to an improved method for introducing a site-directed mutation into a DNA sequence of interest in a target DNA using a single oligonucleotide mutagenic primer which is complementary to the DNA sequence to be mutated in the target DNA and wherein the mutation to be introduced into the DNA sequence by the primer is located within the region of complementarity. In a single reaction, the primer is annealed to the target DNA and extended in an extension/polymerization reaction to produce copies of the target DNA comprising the primer. A selection means is used to remove the target DNA and the copies of the target DNA comprising the primer are propagated in host cells.

In one aspect, the present invention provides a method for introducing a mutation selected from the group consisting of deletion, insertion, substitution, point mutation, and combinations thereof into a DNA sequence of interest, comprising (a) annealing to a target DNA comprising the DNA sequence of interest a mutagenic primer DNA or DNA analog comprising a 5′ end and a 3′ end, each end comprising a DNA sequence complementary to the DNA sequence of interest in the target DNA and wherein the mutagenic primer further includes at least one mutation located between the complementary sequences comprising 5′ and the 3′ ends of the mutagenic primer; synthesizing by means of an extension/polymerization reaction one or more mutated copies of the target DNA comprising the mutagenic primer complementary to the DNA sequence of interest linked to a DNA sequence complementary to the remainder of the target DNA; (c) providing a selection means which selectively removes or degrades the target DNA; and (d) propagating the one or more mutated copies in host cells to produce the DNA sequence of interest with the mutation therein.

In another aspect of the present invention, a method is provided for producing a library of mutations in a DNA sequence of interest, comprising (a) annealing to a target DNA comprising the DNA sequence of interest a plurality of mutagenic primer DNAs or DNA analogs, each comprising a 5′ end and a 3′ end, each end comprising a DNA sequence complementary to the DNA sequence of interest in the target DNA, and each mutagenic primer including at least one mutation located between the complementary sequences comprising 5′ and the 3′ ends of the mutagenic primer; (b) synthesizing by means of an extension/polymerization reaction a plurality of mutated copies of the target DNA, each copy comprising one of the mutagenic primers complementary to the DNA sequence of interest linked to a DNA sequence complementary to the remainder of the target DNA; (c) providing a selection means which selectively removes or degrades the target DNA; and (d) propagating the plurality of mutated copies of the target DNA in host cells to produce the library of mutations in the DNA sequence of interest.

In either one of the above methods, the target DNA is either single-stranded or the target DNA is double-stranded and the mutagenic primers are each complementary to the same strand. Preferably, the target DNA is circular.

In a further aspect of the present invention, a method is provided for introducing at least one mutation into a DNA sequence of interest, comprising (a) providing a double-stranded circular target DNA comprising the DNA sequence of interest and wherein one strand is a template strand and the other strand is a non-template strand; (b) providing a mutagenic primer DNA or DNA analog which includes a 5′ end and a 3′ end, each end comprising a DNA sequence complementary to the DNA sequence of interest in the template strand and wherein the mutagenic primer further includes at least one mutation located between complementary sequences at the 5′ and the 3′ ends; (c) denaturing the double-stranded circular target DNA to separate the template strand from the non-template strand; (d) annealing the mutagenic primer to the template strand to, form an annealed product; (e) subjecting the annealed product to an extension/polymerization reaction using a DNA polymerase to produce an amplified product containing one or more of mutated DNA molecules comprising the mutagenic primer and a DNA sequence complementary to the template strand; (f) using a selection means to remove or degrade the target DNA; and (g) propagating the one or more mutated DNA molecules in host cells to produce the DNA sequence of interest with the at least one mutation.

In a further embodiment of any one of the above methods, the target DNA is methylated either in vitro or in vivo and the selection means is a restriction endonuclease which digests the methylated target DNA. Preferably, the restriction endonuclease is selected from the group consisting of DpnI, MboI, NanII, NmuDI, and NmuEI.

In a further still embodiment of any one of the above methods, the target DNA is methylated either in vitro or in vivo and the selection means is a bacterial host with the ability to digest methylated DNA. Preferably, the bacterial host is a strain of Escherichia coli strain which has a genotype selected from the group consisting of mcr⁺, mrr⁺, and combinations thereof. More preferably, the bacterial host is a strain of Escherichia coli which has an mcrBC⁺genotype.

In a further still embodiment of any one of the above methods, the extension/polymerization reaction is catalyzed by a DNA polymerase which is substantially free of 5′ exonuclease activity and strand displacement activity. In particular embodiments, the extension/polymerization reaction is catalyzed by a DNA polymerase selected from the group consisting of Pfu DNA polymerase, pfx DNA polymerase, Taq DNA polymerase, and modified variants thereof. Preferably, the extension/polymerization reaction is repeated for 35 cycles or less.

In particular embodiments of the above methods, the host cells are procaryotic cells or eucaryotic cells.

In a further still aspect of the present invention, a kit is provided for introducing a specific mutation into a DNA molecule, comprising: (a) a DNA polymerase substantially free of 5′ exonuclease activity and strand displacement activity; (b) an endonuclease which cleaves DNA without the specific mutation; (c) a control DNA molecule; and (d) a control mutagenic primer for the control DNA, wherein the control mutagenic primer includes a 5′ end and a 3′ end, each end comprising a DNA sequence complementary to the control DNA molecule and at least one mutation site located between the complementary 5′ and the 3′ ends.

In particular embodiments of the kit, the endonuclease is selected from the group consisting of DpnI, NanII, NmuDI, and NmuEI and the DNA polymerase is Pfu polymerase. The kit can further include competent cells which in particular embodiments are bacterial cells, preferably a strain of Escherichia coli. Further still, the kit can further include concentrated reaction buffers. In further still embodiments of the kit, the kit further includes computer programs and/or software for determining the melting temperature (Tm) of the mutagenic primers using the nearest-neighbor or other method for determining the Tm.

Objects

Therefore, it is an object of the present invention to provide an improved method for site-directed mutagenesis which can be performed in a single reaction.

It is a further object of the present invention to provide an improved method for site-directed mutagenesis which is efficient, simple to perform, and inexpensive.

These and other objects of the present invention will become increasingly apparent with reference to the following drawings and preferred embodiments.

### DESCRIPTION OF THE DRAWINGS

FIG. 1A is a schematic representation of site-directed mutagenesis using a mutagenic primer (solid arrow) for introducing a point mutation (stars on the solid arrow) into a target DNA. The target DNA is shown as a closed circular single-stranded template nucleic acid (solid circle). The mutated copy of the target DNA is shown by the dotted line. Note that the ends of the mutated copy are not ligated together.

FIG. 1B is a schematic representation of site-directed mutagenesis using a mutagenic primer (solid arrow) for introducing a deletion (gap in solid arrow shown by the vertical arrow) into a target DNA. The target DNA is shown as a closed circular single-stranded template nucleic acid (solid circle). The mutated copy of the target DNA is shown by the dotted line and the nucleic acid deleted from the target DNA is shown by the loop. Note that the ends of the mutated copy are not ligated together.

FIG. 1C is a schematic representation of site-directed mutagenesis using a mutagenic primer (solid arrow) for introducing an insertion (thick line comprising the solid arrow) into a target DNA. The target DNA is shown as a closed circular single-stranded template nucleic acid (solid circle). The mutated copy of the target DNA is shown by the dotted line and the nucleic acid inserted shown by the dotted loop. Note that the ends of the mutated copy are not ligated together.

FIG. 1D is a schematic representation of site-directed mutagenesis using a mutagenic primer (solid arrow) for introducing a substitution (thick line comprising the solid arrow) into a target DNA. The target DNA is shown as a closed circular single-stranded template nucleic acid (solid circle). The mutated copy is shown by the dotted line and the nucleic acid substituted shown by the dotted loop and the nucleic acid replaced by the solid loop. Note that the ends of the mutated copy are not ligated together.

FIG. 2A shows the design of a primer pair (SEQ ID NOs: 5 and 6) for creating a 555 bp deletion of the DNA encoding the GuK functional domain of Pals1 (SEQ ID NO: 29). Shown are the 5′ tail and 3′ head of the primers and their relationship to the Pals1 DNA. The bold-faced type in the Pals1 DNA sequence corresponds to the primer DNA sequences.

FIG. 2B shows the design of a single primer (SEQ ID NO: 5) for creating a 555 bp deletion of the DNA encoding the GuK functional domain of Pals1 (SEQ ID NO: 29). Shown are the 5′ tail and 3′ head of the primer and its relationship to the Pals1 DNA. The bold-faced type in the Pals1 DNA sequence corresponds to the primer DNA sequence.

FIG. 3 shows the design of detection PCR primers for identifying the CT to GC point mutation encoding a single amino acid substitution L25A (leucine to a alanine at codon 25) in the DNA sequence of Pals2 (SEQ ID NO: 30). A single mutagenic primer (SEQ ID NO: 21) was used to introduce the point mutation in the Pals2 DNA. The CT in the Pals2 DNA sequence and the GC in the mutagenic primer are shown in bold-faced type. Forward PCR detection primer (SEQ ID NO: 24) terminating with the GC and a downstream primer (SEQ ID NO: 25) were used for discriminating between bacterial colonies containing the GC and colonies containing the CT as shown in FIG. 4B.

FIG. 4A shows an ethidium bromide stained agarose gel of the PCR amplification products of DNA from bacterial clones which had been transformed with the amplified product from a mutagenesis reaction using two-primers to produce a 555 bp deletion in DNA encoding the Pals1 GuK domain.

FIG. 4B shows an ethidium bromide stained agarose gel of the PCR amplification products of DNA from bacterial clones which had been transformed with the amplified product from a mutagenesis reaction using a single primer to produce a point mutation in DNA encoding the Pals2 L25A domain.

FIG. 4C shows an ethidium bromide stained agarose gel of the PCR amplification products of DNA from bacterial clones which had been transformed with the amplified product from a mutagenesis reaction using a single primer to produce an insertion of Myc-tag DNA into DNA encoding Crumbs3.

### DETAILED DESCRIPTION OF THE INVENTION

All patents, patent applications, government publications, government regulations, and literature references cited in this specification are hereby incorporated herein by reference in their entirety. In case of conflict, the present description, including definitions, will control.

The present invention provides an improved site-directed mutagenesis method which enables not only the introduction of point mutations into a DNA sequence of interest but also enables a wide size range of insertions, deletions, and substitutions to be made in the DNA sequence of interest. The key elements of the method include introducing a mutation into the sequence of interest in a target DNA using a single oligonucleotide mutagenic primer, which in an extension/polymerization reaction, produces one or more newly synthesized copies of the target DNA comprising the primer with the mutation. Preferably, the reaction is cyclic which produces a multiplicity of the mutated copies of the target DNA. A selective means is used for degrading or removing the parental target DNA from the newly synthesized copies of the target DNA with the mutation and propagation of the copies in host cells. Thus, the method comprises three steps (1) design and synthesis of the primer, (2) annealing the primer to a parental target DNA and producing one or more mutated copies of the parental target DNA in a single reaction, and (3) degrading or removing the parental target DNA and propagating the mutated copies in host cells.

Thus, in the first step, an oligonucleotide mutagenic DNA or DNA analog primer is prepared for introducing a mutation into a DNA sequence of interest in a circular target DNA (parental DNA). The mutagenic primer comprises a DNA sequence which is complementary to the DNA sequence of interest in the target DNA. Included within the complementary DNA sequence of the mutagenic primer is a DNA sequence which is to be inserted or substituted into the DNA sequence of interest in the target DNA or the one or more nucleotide to be changed in the sequence of interest in the target DNA. The DNA sequence to be inserted into the target DNA sequence of interest is placed with the mutagenic primer such that it is flanked by the complementary DNA sequences. In the case of a mutagenic primer for introducing a deletion into the sequence of interest in the target DNA, the complementary DNA sequences comprising the mutagenic primer are complementary to the DNA sequences in the DNA sequence of interest in the target DNA which flank the DNA sequence which is to be deleted.

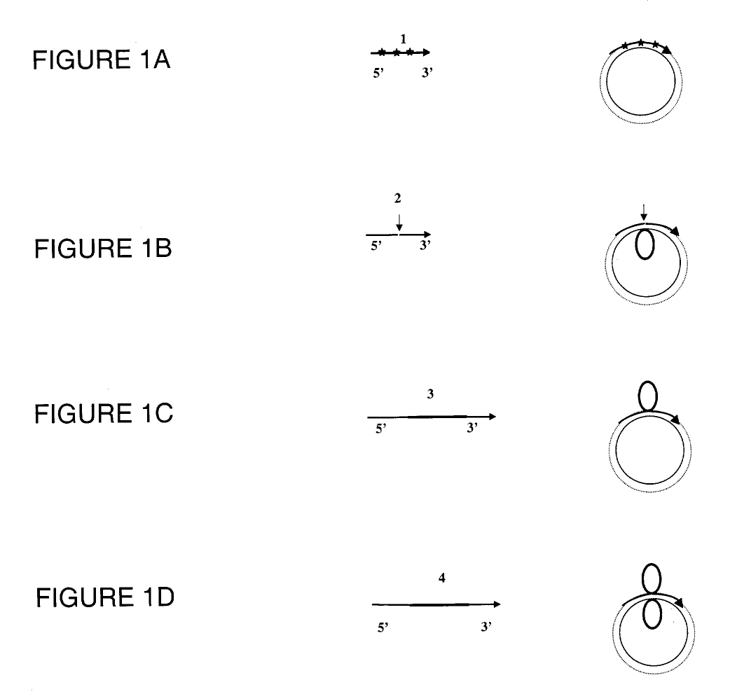

After the mutagenic primer has been prepared, the mutagenic primer is annealed to the DNA sequence of interest in the target DNA. In the case for introducing one or more point mutations in the DNA sequence of interest, the complementary primer sequences flanking the nucleotides for the one or more point mutations are annealed to the target DNA sequence of interest to produce a mismatch between the nucleotides in the primer for the point mutation and the corresponding nucleotides in the DNA sequence of interest (FIG. 1A). In the case for introducing an insertion into the DNA sequence of interest, the complementary primer sequences flanking the DNA sequence to be inserted into the DNA sequence of interest are annealed to the DNA sequence of interest in the target DNA such that the DNA sequence to be inserted into the DNA sequence of interest, which cannot anneal with the DNA sequence of interest, forms a loop (FIG. 1B). In the case for introducing a deletion into a DNA sequence of interest, the complementary primer sequences are annealed to the DNA sequence of interest in the target DNA which flank the DNA sequence to be deleted. This forms a loop of non-annealed DNA comprising the DNA to be deleted in the DNA sequence of interest (FIG. 1C). In the case for substituting a DNA sequence in the target DNA with another DNA sequence, the complementary primer sequences flanking the DNA sequence to be exchanged in the DNA sequence of interest are annealed to the DNA sequence of interest in the target DNA. Because the DNA sequences to be exchanged are non-complementary, a bubble is formed at the region of non-complementarity (FIG. 1D).

After the mutagenic primer has been annealed to the DNA sequence of interest in the target DNA, the 3′ end of the mutagenic primer is extended in an extension/polymerization reaction with a DNA polymerase to produce a substantially full-length mutated complementary copy of the target DNA which comprises the mutagenic primer covalently linked at its 3′ end to the complementary copy of the remainder of the target DNA in a double-stranded duplex with the target DNA. In other words, the double-stranded DNA product comprises the mutated copy of the target DNA as a first strand and the target DNA as the second strand. Preferably, the extension/polymerization reaction comprises an excess of mutagenic primer and is cyclic. Therefore, after the double-stranded DNA product is produced, the double-stranded DNA product is denatured and the target DNA allowed to anneal with additional mutagenic primer and the mutagenic primer extended/polymerized as above. This cyclic process is repeated to produce an amplified product comprising a multiplicity of mutated copies of the target DNA, each copy comprising a mutagenic primer linked at its 3′ end to a complementary copy of the target DNA.

Then, without further treatment of the mutated copies, for example, treating the mutated copies with a DNA ligase, a selection means is used to degrade or remove the target DNA from the mutated copies of the target DNA. Therefore, the amplified product is treated in a reaction with the selection means which selectively degrades or removes the target DNA. The mutated copies of the target DNA which remain after selection are propagated in procaryotic or eucaryotic host cells.

The degradation of the target DNA can be achieved either by degrading or digesting the target DNA in vitro with a selective enzyme which recognizes only the target DNA prior to propagating the multiplicity of mutated copies of the target DNA in host cells or by propagating the amplified product in host cells which are capable of selectively degrading or digesting the target DNA before or while it propagates the multiplicity of mutated copies of the target DNA.

The removal of the target DNA can be achieved as follows. The mutagenic primer is reversibly bound to a solid support comprising a polymer, glass, paramagnetic particles, or the like. For example, the mutagenic primer can be synthesized bound to a solid support using methods commonly used for synthesizing oligonucleotides. The reversibly bound mutagenic primer is used to make mutated copies of the target DNA as described herein. In a preferred embodiment, after the extension/polymerization reaction, the mutated copies of the target DNA are removed from the solid support and propagated in host cells, preferably, host cells which preferentially degrade the target DNA. In particular embodiments, the amplified product comprising the mutated copies of the target DNA, which are bound to the solid support via the mutagenic primer, is denatured with heat or an alkali and the target DNA selectively degraded or in some embodiments, removed by washing either by column chromatography or in batch. Afterwards, the mutated copies of the target DNA are removed from the solid support and propagated in host cells.

While the above methods can be applied to circular double-stranded target DNAs or circular single-stranded target DNAs, it is envisioned that the method will be used primarily to mutate target DNAs which are double-stranded simply because most cloned DNA sequences of interest are for matters of convenience, propagated in double-stranded plasmids or vectors. Whether the target DNA is single-stranded or double-stranded, the production of mutated copies of the target DNA is performed in a single reaction.

In the case of a circular double-stranded target DNA, the mutagenic primer is complementary to only one of the strands of the target DNA. Thus, the target DNA comprises a parental template strand which is complementary to the mutagenic primer and a parental non-template strand which is not complementary to the mutagenic primer. When the target DNA comprising the DNA sequence of interest comprises a circular double-stranded plasmid cloning vector, denaturation of the double-stranded circular target DNA produces a closed circular single-stranded template strand and a closed circular single-stranded non-template strand. In general, circular double-stranded DNAs such as plasmids and the like exist primarily as supercoiled DNA molecules of which a sub-population exists as relaxed circular DNA molecules. The relaxed circular DNA molecules are the result of one or more nicks in one or the other or both strands of the DNA molecule which enable the supercoiled DNA molecule to unwind to form an open double-stranded circle. When a preparation of circular double-stranded DNA is denatured, the following products are believed to be produced: closed circular DNA molecules in which both strands are intertwined, closed circular single-stranded DNA molecules consisting of one or the other of the strands, and linear DNA molecules consisting of one or the other of the strands or fragments thereof. The closed circular single-stranded DNA molecules comprising the template strand are believed to serve as the template in the method of the present invention.

In the reaction, just as described above, the target DNA is denatured and the mutagenic primer is annealed to the complementary strand. After the mutagenic primer has been annealed to the template strand, the 3′ end of the mutagenic primer is extended in an extension/polymerization reaction with a DNA polymerase to produce a substantially full-length mutated complementary copy of template strand which comprises the mutagenic primer covalently linked at its 3′ end to the complementary copy of the template strand in a double-stranded duplex with the template strand. The non-template strand is not believed to participate in the reaction. Preferably, the extension/polymerization reaction comprises an excess of mutagenic primer and is cyclic. Therefore, after the double-stranded DNA product is produced, the double-stranded DNA product is denatured and the template strand allowed to anneal with additional mutagenic primer and the mutagenic primer extended/polymerized as above. This cyclic process is repeated to produce an amplified product comprising a multiplicity of mutated copies of the template strand, each copy comprising a mutagenic primer linked at its 3′ end to a complementary copy of the template strand.

A selection means as discussed herein is then used to remove or degrade the template strand and, optionally, the non-template strand from the mutated copies of the template strand. The amplified product is treated in a reaction with the selection means which selectively destroys or removes the template strand and optionally the non-template strand. The multiplicity of mutated copies of the template strand which remain after selection are propagated in procaryotic or eucaryotic host cells.

The present invention provides a number of significant advantages over current methods for performing site-directed mutagenesis. These advantages include universality because the method combines all site-directed mutagenesis applications into a single platform, robustness and high efficiency, simplified experimental design, reduced hands-on time, and reduced cost since only one primer is needed for the mutagenesis. In addition, the single primer of the present invention allows more flexibility in controlling primer concentration in the polymerization step unlike using two partially or fully complementary mutagenic primers which can form dimer pairs which reduces the effective concentration of each primer in an uncontrollable manner. Also, unlike many other site-directed mutagenesis methods, the present method does not require a primer with a phosphorylated 5′ end because the method does not require a ligation step following the amplification reaction to ligate the ends of the mutated copies of the target DNA to form closed circular molecules for propagation in host cells. Furthermore, the method is readily adaptable to be used in a multiplex embodiment for producing a library of mutations in a target DNA sequence of interest. In the multiplex embodiment, a plurality of mutations can be generated in a DNA sequence of interest in a target DNA simultaneously by including in the reaction disclosed herein a plurality of mutagenic primers, each comprising a particular mutation. Each of the mutagenic primers are preferably all complementary to the template strand as above. The extension/polymerization reaction produces a plurality of mutated copies of the target DNA which as described previously can be propagated in host cells to produce the library. The host cells can then be screened using methods well known in the art for identifying particular mutants. The multiplex method is particularly useful in proteonomics where it is necessary to produce a plurality of DNAs in which particular DNA sequences encoding particular domains of a protein are substituted with other sequences, deleted, or rearranged.

In light of the above, the method of the present invention is suitable for a wide variety of applications including, but not limited to, combinatorial proteonomics research (domain editing and/or swapping), immunology, cell biology and imaging (protein tag insertion), pharmacogenomics, and drug discovery. For example, the method can be used for introducing multiple mutations into a single cloned sequence simultaneously. The method allows multiplexing different mutations in the same tube with different primers annealing to different targets with subsequent segregation. The method enables screening of mutant libraries. That is, the method enables introducing and screening for multiple mutations in a protein of interest in search for particular phenotype. A set of mutagenic primers are mixed with the cloned protein DNA sequence in a single tube, mutagenesis is performed as taught herein, and transformed host cells are screened for desired phenotype. This application is only possible with sets of primers which do not have complementarity to each other as would occur in the two-primer methods. The mutagenesis reaction can be performed with the one or more mutagenic primers immobilized on microtiter plates for high throughput applications. In addition, some pharmaceutical companies are now using kilobase-long synthetic DNA sequences for optimization of expression (editing the amino acid codons and control regions) . The cost of synthesis for these molecules is 10 times higher per base as compared to oligonucleotides. With the method of the present invention it is possible to use these long single-stranded sequences directly without any additional biochemical procedures in insertional mutagenesis (for cloning purposes) saving thousands of dollars per gene sequence. Furthermore, cloning may be accomplished without restriction endonucleases and ligation using insertions or substitutions as exemplified by the Myc insertion and the domain substitution shown in the Examples. The method of the present invention also enables mutagenesis of viral DNA and chromosomal DNA to be made such as gene introduction into a heterologous system as well as gene knockout.

The mutagenic primer used to introduce the mutation into the target DNA comprises three general regions. The first region (5′ tail) comprises a DNA sequence at the 5′ end of the primer which is complementary to a DNA sequence comprising the DNA sequence of interest in the template strand of the target DNA which is upstream of and adjacent to the site where the mutation is to be introduced. The second region (3′ head) comprises a DNA sequence at the 3′ end of the primer which is complementary to a DNA sequence comprising the DNA sequence of interest in the template strand of the target DNA which is upstream of and adjacent to the site where the mutation is to be introduced. The 3′ end of the tail serves to prime the extension/polymerization reaction which produces the mutagenized copy of the template strand. The third region (internal site) which is located between the first region and the second region comprises the mutation to be introduced into the DNA sequence of interest in the target DNA.

For producing a point mutation in the DNA sequence of interest, the internal site of the mutagenic primer comprises one or more nucleotides which are to replace the one or more nucleotides in the DNA sequence of interest in the target DNA. For producing an insertion or substitution in the DNA sequence of interest, the internal site of the mutagenic primer comprises the DNA sequence to be inserted or substituted into the sequence of interest. For producing a deletion in the DNA sequence of interest, the mutagenic primer comprises a 5′ tail and 3′ head and the internal site is null. In this primer, the DNA sequences comprising the 5′ tail and 3′ head are complementary to the DNA sequences in the DNA sequence of interest which flank the DNA sequence to be deleted.

In further embodiments of the mutagenic primer, the internal site can comprise more than one mutation, each mutation separated by DNA sequences complementary to the DNA sequence in the DNA sequence of interest.

In practicing the site-directed mutagenesis method of the present invention, it is important that the mutagenic primer's melting temperature (Tm) be accurately determined. Tm determinations for mutagenic primers for introducing point mutations are based on the entire length of the mutagenic primer and takes into consideration the nucleotide mismatches at the one or more sites of the target DNA to be mutated. However, for Tm determinations for the mutagenic primers for introducing insertions, substitutions, or deletions, a Tm is separately determined for the first and second regions. The calculations for determining the Tm of the mutagenic primers can use any of the methods well known in the art. A particularly suitable method is the nearest-neighbor algorithm method. Preferably, the complementary sequences comprising the first and second regions comprise about 40% G+C or greater. The nucleotide length of the first and second regions of the mutagenic primer are preferably between about 10 to 50 nucleotides in length, more preferably, between about 15 to 30 nucleotides in length. However, in particular embodiments, it may be necessary to use a mutagenic primer wherein the nucleotide length of the first or second regions, or both, is greater than 50 nucleotides in length so as to obtain the mutagenesis result desired. The nucleotide length of the first and second regions of the mutagenic primers may be the same or may be different lengths. Thus, the mutagenic primers can include 5′ tails and 3′ heads of any nucleotide length, can contain any heterologous DNA sequence in the internal site of any nucleotide length, can introduce deletions of any length, and can be produced using any of the known methods for producing DNA primers such as making the mutagenic primer by PCR and rendering it single-stranded by methods well known in the art such as using a biotinylated nucleotide at the 5′ end of one strand and capturing the biotinylated strand by strep-avidin capture. In a further embodiment, the mutagenic primer can comprise the complementary 3′ head linked at its 5′ end to the DNA sequence to insert or exchange in the target DNA instead of the complementary 5′ tail. Sambrook et al. (Molecular Cloning: A Laboratory Manual, Second Edition, Cold Spring, Cold Spring Harbor, N.Y. (1989)) provide methods for designing mutagenic primers for site-directed mutagenesis of cloned DNA which can be followed for designing the mutagenic primers described above.

In particular embodiments, the mutagenic primer can be 5′ phosphorylated. The phosphorylation can be by any of the methods well known to a person of ordinary skill in the art such as T-4 polynucleotide kinase treatment or the like. Preferably, after phosphorylation, the phosphorylated mutagenic primer is purified prior to use to remove contaminants which might interfere with the mutagenesis method. Preferred purification methods are fast high-pressure liquid chromatography (HPLC) or polyacrylamide gel electrophoresis; however, other purification methods may be used. In general, these purification steps are unnecessary when a non-phosphorylated mutagenic primer is used. While a 5′ phosphorylated mutagenic primer is not necessary to practice the invention, in some instances a 5′ phosphorylated mutagenic primer may facilitate the ligation of the 5′ and 3′ ends of the mutagenized copy of the template DNA following transformation into the microorganism. Furthermore, a 5′ phosphorylated mutagenic primer is useful when it is desirable or advantageous to ligate the 5′ and 3′ ends of the mutated copies of the target DNA to produce closed circular molecules.

The preferred method for producing the mutated copies of the DNA sequence of interest in the target DNA is a cyclic extension/polymerization reaction such as the linear cyclic amplification reaction described in U.S. Pat. Nos. 5,789,166; 5,932,419; and, 6,391,548, all to Baeur et al. Linear cyclic amplification is a derivative PCR method which differs from PCR in that the amplification of the target DNA is linear and not exponential. For exponential amplification of a target DNA, the reaction contains opposing primers which flank the DNA sequence of interest to be amplified in a double-stranded target DNA. The opposing primers enable the synthesis of both strands comprising the DNA sequence of interest which under cyclic reaction conditions results in the exponential amplification of the DNA sequence of interest. In contrast, in the present invention, the single mutagenic primer and the single-stranded target DNA comprising the DNA sequence of interest allow synthesis of only a DNA strand which is complementary to the target DNA which under cyclic reaction conditions results in a linear amplification of the target DNA, that is, multiple copies of only one of the two strands comprising the target DNA.

The exact parameters for each cycle of the cyclic amplification reaction can vary in accordance with factors such as the DNA polymerase used, the GC content of the mutagenic primer, concentration of the mutagenic primer and the target DNA, and the like. The most important parameters, however, include the time for the denaturation, annealing, and extension/polymerization steps and the temperature at which each of these steps takes place. Publications describing polymerase chain reactions (PCR) can provide useful guidance for determining the correct parameter for each step of the cyclic amplification reaction. For example, the following U.S. Patents, which all relate to PCR, can provide useful guidance: U.S. Pat. Nos. 4,683,195, 4,800,159, and 4,965,188, all to Mullis et al.; U.S. Pat. No. 4,683,202 to Mullis; U.S. Pat. Nos. 4,889,818 and 5,079,352, both to Gelfand et al.; U.S. Pat. Nos. 5,075,216 and 5,091,310, both to Innis et al.; U.S. Pat. No. 5,104,792 to Silver et al.; U.S. Pat. No. 5,023,171 to Ho et al.; and U.S. Pat. No. 5,066,584 to Gyllensten et al. Regardless of the particular reaction parameters chosen, the cyclic extension/polymerization step should proceed for a length of time sufficient to produce a single-stranded mutagenized copy of the target DNA which is substantially equivalent in length (excluding insertions, substitutions or deletions introduced by the mutagenic primer) to the entire length of the target DNA.

In the present invention, it has been found to be preferable that the annealing temperature be about 20 to 10° C. higher than the Tm of the 3′ head of the mutagenic primer. This has been found to increase the specificity of the extension/polymerization step which can be performed at the annealing temperature or at a higher temperature. In the present invention, it is also preferable that the number of amplification cycles be less than 35 cycles. More preferably, the number of amplification cycles should be less than 20 cycles. However, it is to be understood that the actual number of cycles for any particular mutagenesis will depend on the number, complexity, size, and type of mutations to be introduced and the amount of target DNA in the reaction. Therefore, in particular cases, the number of cycles may exceed 35 cycles. In other cases, one cycle may produce a sufficient number of mutated copies of the target DNA. Thus, for any particular amplification, the optimal conditions will be determined empirically.

The preferred DNA polymerases for the extension/polymerization step are high fidelity DNA polymerases which lack the ability to displace the mutagenic primer annealed to the target DNA and which do not have substantial 5′ exonuclease activity. It is preferable that the polymerase have 3′ exonuclease proofreading activity. The 3′ exonuclease proofreading ability enables mutated copies of the template strand to be produced which are essentially the same length as the template strand and which are unlikely to contain spurious mutations introduced during the extension/polymerization step. It is further preferable that the polymerase used in the reaction be a thermostable polymerase. A thermostable polymerase facilitates the production of multiple mutated copies of the target DNA in a cyclic extension/polymerization reaction. The polymerase may be isolated from naturally occurring cells or may be produced by recombinant DNA technology. The thermostable Pfu DNA polymerase (commercially available from Stratagene, La Jolla, Calif.) is an example of a suitable polymerase for the extension/polymerization reaction. Examples of other DNA polymerases which are suitable include, but are not limited to, phage T7 polymerase, phage T4 polymerase, VENT and DEEP VENT DNA polymerases (commercially available from New England Biolabs, Beverly, Mass.), and recombinant polymerases such as PLATINUM Pfx polymerase or PLATINUM Taq polymerase (both commercially available from Invitrogen, Carlsbad, Calif.). When the template strand for mutagenesis is relatively long, it may be desirable to use a mixture of thermostable DNA polymerases. For example, for some template strands it has been found to be useful to use a mixture of DNA polymerases wherein one of the DNA polymerases has 5′ exonuclease activity and the other DNA polymerase lacks 5′ exonuclease activity (See U.S. Pat. No. 5,436,149 to Barnes for example).

The selection means for removal of the parental target DNA from the newly synthesized mutated copies of the target DNA can be effected either by digesting the amplified product produced in the extension/polymerization reaction with a selective enzyme which selectively degrades the parental target DNA in an in vitro step prior to transforming host cells, by a non-enzymatic method such that discussed above where the mutagenic primer is reversibly bound to a solid support, or in vivo by transforming the amplified product directly into host cells which have the ability to selectively degrade the parental target DNA. A practical method for selectively removing the parental target DNA is to take advantage of the fact that most target DNAs have been propagated in a host cell which methylate various nucleotide residues in the target DNA, that synthesizing DNAs in vitro produces DNAs which are not methylated, and that there are restriction endonucleases or host cells which will selectively digest methylated DNA while not substantially digesting the unmethylated DNA. For DNAs which have not been propagated in host cells which methylate DNA, the target DNA can be methylated in vitro prior to performing the annealing step to render the parental target DNA digestible following the extension/polymerization reaction. In vitro methylation can be achieved in a reaction which uses a DNA methylase such as the Dam methylase in the presence of S-adenosylmethionine (SAM) to methylate adenosine residues in the sequence GATC which renders the target DNA digestible with restriction endonucleases such as DpnI, NanII, NmuDI, and NmuEI, and the like. Alternatively, the DNA is methylated with the SssI methylase which methylates cytosine residues in the sequence GC which renders the parental target DNA digestible with a restriction endonuclease which only cleave a recognition sequence in which the cytosine residue is methylated. While the methylated DNA method is preferred, there are other selection methods which may be used. For example, the mutagenized copies of the target DNA are synthesized during the extension/polymerization reaction using particular DNA analogs which when incorporated into the mutagenized copies, enable the mutagenized copies to resist digestion with a selective enzyme which digests DNA but which is substantially inhibited from digesting DNA which contains the analog.

In an in vitro method for selectively removing the parental target DNA, a preferred selective enzyme is a restriction enzyme such as DpnI which only cleaves DNAs in which the adenosine residue in the recognition sequence GATC is methylated. In general, most cloned DNAs are propagated in Escherichia coli strains which methylate the adenosine residues of the GATC sequence. Thus, the double-stranded target DNA is capable of being digested with DpnI whereas the newly synthesized mutagenized copies of the target DNA, which are not methylated, are resistant to digestion. There are conflicting descriptions of DpnI's template specificity. Some reports suggest that DpnI only digests GATC sequences in which the adenosine residues of both strands are methylated whereas other sources suggest that DpnI can also digest GATC sequences in which only one of the adenosine residues is methylated (hemimethylated). In the case of hemimethylated GATC sequences, some reports suggest that both strands are cleaved whereas others suggest that only the methylated strand is cleaved. It is generally accepted that DpnI does not digest single-stranded methylated DNAs. Without intending to be bound by any theory, it is believed that DpnI digests both strands of fully methylated DNAs and only the methylated strands of hemimethylated DNAs but may not digest methylated single-stranded DNAs.

Alternatively, instead of using a restriction endonuclease to selectively remove the parental target DNA, a method such as the uracil N-glycosylase method first described in Kunkel, Proc. Natl. Acad. Sci. USA, 82:488-492 (1985) can be used. In this case, the double-stranded target nucleic is propagated in a bacterial strain which substitutes the thymidine residues in the DNA with uracil residues. This renders the template and non-template strands susceptible to digestion with uracil N-glycosylase whereas the newly synthesized mutagenized copies, which do not contain uracil residues in place of the thymidine residues, are not digested. The digestion can be performed in vitro by adding the uracil N-glycosylase to the amplified product following the extension/polymerization step or in vivo by transforming the amplified product into a bacterial strain with the uracil N-glycosylase therein to degrade template and non-template strands.

In an in vivo embodiment for selectively removing the parental target DNA, the target DNA is methylated in vitro or by propagation in a host cell with the ability to methylate the DNA. Host cells which methylate DNA transformed therein are well known to those skilled in the art. After, the extension/polymerization reaction step, the entire reaction mixture is transformed into an Escherichia coli strain with a functional methylase restriction system, that is, an Escherichia coli strain which is mcr⁺, mrr⁺, or both. An example of a suitable mcr⁺strain is DH5 which has an mcrBC⁺genotype. In the Escherichia coli, the target DNA is digested and mutagenized double-stranded copies of the target DNA are produced. This embodiment avoids the previously described in vitro digestion step or synthesis of target DNA with the thymidine residues replaced with uracil residues. Furthermore, one skilled in the art would understand that other combinations of methylase systems could be used in lieu of the above systems.

In general, the preferred host cells for transforming with the amplified product are procaryotic cells such as the various strains of Escherichia coli which have been rendered competent for transformation. In particular embodiments, it may be desirable to transfect the amplified product into a eucaryotic cell. Methods for preparing and transforming competent procaryotic cells or transfecting eucaryotic cells are well know in the art and can be found, for example, in Sambrook et al., Molecular Cloning: A Laboratory Manual Coldspring Harbor Press, Coldspring Harbor, N.Y. (1989), and the like.

A typical mutagenesis reaction of the present invention is performed in a reaction mixture having a volume ranging from between about 5 to 100 μL. The reaction mixture preferably contains about 5 to 300 ng of target DNA, or in particular embodiments, more than 300 ng of target DNA, about 50 to 500 nM of mutagenic primer, about 100 to 500 μM deoxynucleotide triphosphates (dNTP), and about 0.5 to 10 units of DNA polymerase in a reaction buffer. The reaction buffer preferably has a buffering capacity which is operative at a physiological pH such as a pH between about 6 and 9 (inclusive) and include at least 1 to 20 mm of a salt such as NaCl or KCl and 1 to 50 mM magnesium, preferably in the form of MgCl or MgSO₄. Optionally, the reaction buffer can further include one or more of a protein, preferably at a concentration of about 0.1% (w/v), a detergent or surfactant, preferably between about 0.1 to 2%, and the like. A reaction buffer which has been used with Pfu DNA polymerase comprises about 10 mM KCl, 10 mM (NH₄)₂SO₄, 20 mM Tris-HCl (pH 8.8), 20 mM MgSO₄, 1% TRITON X-100, and 1 mg/mL nuclease-free bovine serum albumen.

Typical reaction conditions include an initial denaturation step of between about 0.1 and 5 minutes at between about 94° to 98° C. (inclusive). Followed by 1 to 35 cycles, of extension/polymerization. Each cycle comprising about 5 to 60 seconds of denaturation at a temperature between about 94° to 98° C. (inclusive), about 5 seconds to 1 minute of annealing at a temperature at or about 2° C. greater than the Tm of the mutagenic primer, and about 1 to 20 minutes of extension/polymerization at a temperature at or about 20° C. greater than the Tm of the mutagenic primer, or at a higher temperature such as a temperature between about 65° C. and 75° C. (inclusive). Optionally, the finial cycle of the extension/polymerization reaction can include a shorter or longer extension time. The target DNA can be removed and the mutated copies of the target DNA propagated as described previously. Analysis of the mutated copies of the target DNA uses methods well known in the art for analyzing DNA.

Another aspect of the present invention is to provide kits for performing the site-directed mutagenesis method of the present invention. The kit provides one or more of the enzymes or other reagents for use in performing the method. The kit may also contain reagents in pre-measured amounts so as to ensure both precision and accuracy when performing the method. Preferably, the kit also contains instructions for performing the method of the present invention. In a preferred embodiment, the kit comprises a polymerase as discussed above, an endonuclease which digests the target DNA (DNA without the specific mutation) as discussed above, at least one control primer, and a control single-stranded or double-stranded target DNA, preferably the control target DNA is circular. Preferably, the polymerase substantially lacks 5′ exonuclease activity and strand displacement activity. Optionally, the kit can also contain one or more of the following: individual nucleotide triphosphates, individual nucleotide triphosphate analogs, or various mixtures thereof; one or more methylases for in vitro methylation; bacterial strains for propagating the double-stranded target DNAs; frozen competent cells; concentrated reaction buffers; a computer program and/or software with an algorithm for determining primer Tms, for example, the nearest-neighbor algorithm, and the like. In particular embodiments of the kit, the kit further includes frozen competent cells which are capable of degrading the target DNA such as bacterial strains which are mcr⁺ or mrr⁺, or both, for example, DH5 cells with the mcrBC⁺ genotype. In further particular embodiments of the kit, the kit includes only the above frozen competent cells which are capable of degrading the target DNA.

The following examples are intended to promote a further understanding of the present invention.

### EXAMPLE 1

This example illustrates the improved method of the present invention for generating deletion and point mutants and compares the efficiency of the method of the present invention to an amplification-based site-directed mutagenesis method which uses two primers for the mutagenesis. The method of the present invention enables the use of a single primer to generate deletions up to an exceeding 3,000 bp and point mutations in a single amplification reaction.

In our laboratory, site-directed mutagenesis is routinely performed using the QUIKCHANGE site-directed mutagenesis kit (Stratagene, La Jolla, Calif.) for point mutations. It had been reported that the QUIKCHANGE mutagenesis kit was useful only for introducing into a target DNA deletions or insertions of only 12 bp in length (Papworth et al., Strategies 9: 3-4 (1996)). Thus, it was not clear how the kit could be used to introduce deletions of more than 12 bp in length (Papworth et al., Strategies 9: 3-4 (1996)). We used a plasmid containing Pals1 cDNA (SEQ ID NO: 29; total length 6.7 kb; GenBank Accession No. AF199008) as a template to make several deletion and point mutants (Kamberov et al. J. Biol. Chem. 275: 11425-11431 (2000)). The mutagenesis and extension/polymerization reaction was performed in a reaction volume of 25 μL that included 1 μL plasmid DNA (in TE or water) prepared using a WIZARDPLUS SV Miniprep DNA Purification System (Promega, Madison, Wis.) or a QIAPREP Spin miniprep kit (Qiagen, Valencia, Calif.) miniprep containing about 100-300 ng/μL template DNA, 200 nM each primer, 200 μM mixture of deoxynucleotide triphosphates (dNTP) and 1.25U Pfu polymerase in 1×Pfu DNA polymerase reaction buffer.

A preliminary step of denaturation at 95° C. for 3 min was followed by 18 cycles of extension/polymerization. These PCR cycles consisted of 15 seconds of denaturation at 950° C., 1 minute of annealing at a temperature 2° C. higher than the melting Tm of the head of the primer to increase specificity of amplification (see below) and 12 minutes of extension at 68° C. using a PTC-200 thermal cycler (MJ Research, Watertown, Mass.). In cases when the Tm exceeded the extension temperature, a two-step extension/polymerization at 68° C. was performed. After the extension/polymerization reaction, the amplified product was treated with 1 μL of DpnI endonuclease for 2 hours at 37° C. and 2 μL of the DpnI-digested DNA was transformed into 50 μL EPICUREAN COLI XL1 Blue supercompetent cells. The number of kanamycin-resistant clones varied from 100 to 2000.

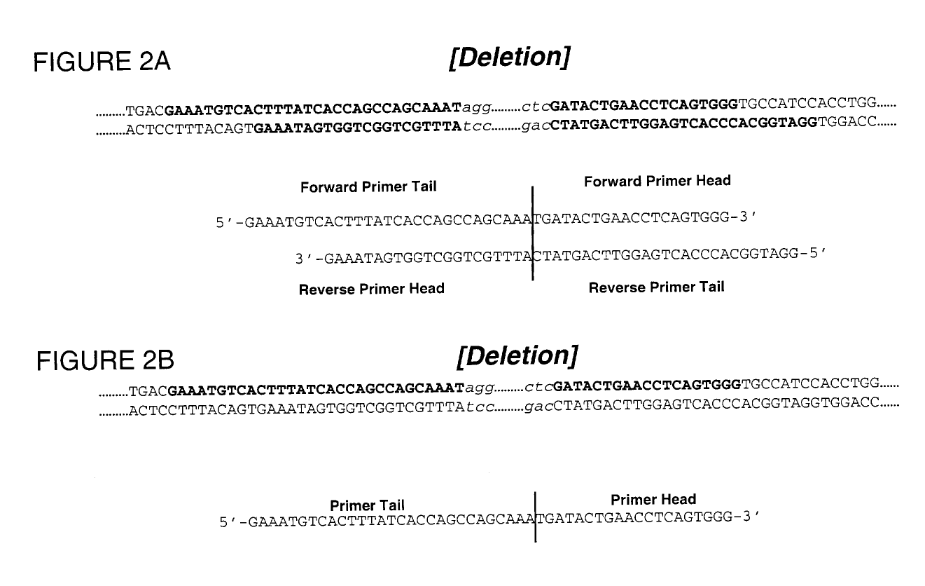

Various primer pairs (Table 2) were tested to determine which would produce the highest yield of the desired deletion. Clones were routinely analyzed by PCR using primers flanking the deletions and efficiencies of between 25% and 70% were observed. Various clones were sequenced to confirm the PCR results. Part A of Table 1 summarizes different examples of primer pairs used in the mutagenesis experiments and the efficiencies for making the various mutants. Table 2 provides the DNA sequences of the primers which were used. For simplicity, the 5′ end part of the primers complementary to the region upstream of the introduced deletion was called the 5′ tail, and the 3′ end part of the primer complementary to the region down-stream of the deletion was called the 3′ head. FIG. 2A show an example of a primer pair for making a 555 deletion in the GuK domain of a target DNA encoding Pals1.

As shown in Part A of Table 1, when using primer pairs of different design, deletions of up to and exceeding 3 kb could be introduced into the plasmid by this simple one-step protocol. The results indicated that the same approach could be used for any DNA sequence because we had successfully deleted several regions of the coding sequence of the plasmid corresponding to different functional domains in the target protein. Deletions of up to 3,184 bp in length were made without any apparent decrease in efficiency. This implies that there may be no practical limitation to the size of deletion or truncation that can be generated.

TABLE 1
Summary of the Mutagenesis Experiments

| Primer | | | | Size of |
|---|---|---|---|---|
| SEQ ID | Primer Length | Tm of Tail | Observed | Mutation |
| NO: | Head and Tail^{a} | And Head^{b} | Efficiency^{c} | (bp) |
| | (A) Primer Pairs | | | |
| 1 | Forward 27 + 27 | 59 + 72 | 25% | 225 |
| 2 | Reverse 27 + 27 | 72 + 59 | | |
| 3 | Forward 24 + 24 | 69 + 64 | 50% | 132 |
| 4 | Reverse 24 + 24 | 58 + 75 | | |
| 5 | Forward 30 + 19 | 67 + 49 | 60% | 555 |
| 6 | Reverse 27 + 21 | 68 + 55 | | |
| 7 | Forward 12 + 23 | 26 + 63 | 30% | 555 |
| 8 | Reverse 17 + 25 | 37 + 63 | | |
| 9 | Forward 30 + 23 | 65 + 54 | 60% | 3184 |
| 10 | Reverse 27 + 25 | 64 + 56 | | |
| 11 | Forward 16 + 27 | 32 + 64 | 70% | 3184 |
| 12 | Reverse 17 + 28 | 62 + 42 | | |
| 13 | Forward 21 + 21 | 55 + 54 | 25% | 555 |
| 14 | Reverse 21 + 21 | 55 + 54 | | |
| 15 | Forward 15 + 19 | 28 + 50 | 70% | 174 |
| 16 | Reverse 16 + 24 | 53 + 41 | | |
| | (B) Single | | | |
| | Primers Used for | | | |
| | Deletions | | | |
| 13 | 21 + 21 | 55 + 54 | 50% | 555 |
| 17 | 22 + 16 | 57 + 57 | 50% | 60 |
| 18 | 16 + 20 | 48 + 48 | 30% | 183 |
| 19 | 18 + 17 | 45 + 46 | 50% | 204 |
| | (C) Single | | | |
| | Primers Used for | | | |
| | Point Mutations | | | |
| 20 | 19 + 21 | 45 + 53 | 70% | 2 |
| 21 | 16 + 14 | 32 + 33 | 66% | 2 |
| | (D) Single | | | |
| | Primer Used for | | | |
| | Insertion | | | |
| 22 | 22 + 23 | 60 + 57 | 50% | 30^{d} |
| | (E) Single | | | |
| | Primer Used for | | | |
| | Substitution | | | |
| 23 | 49 + 50 | 50% | 174 | |
| | | | | subst. |
| | | | | For 9^{e} |

^{a}Primer Sequences are listed in Table 2.
^{b}For definition of 5′ tail and 3 head see FIGS. 2A and 2B. Tm was calculated using Primer Premier software.
^{c}Efficiency was calculated by analyzing at least 10 clones by PCR with analytical primers flanking the mutation, taken as percentage of mutant clones vs the total number of colonies obtained. Selected clones were verified by sequencing.
^{d}Myc-tag (30 bp) was inserted into Crumbs3 protein cDNA sequence.
^{e}A functional domain (L27N) of PALS 2 protein (174 bp) was substituted with the recognition site for PmeI restriction endonuclease (9 bp).

TABLE 2
Oligonucleotide Primer Sequences*

| | |
|---|---|
| Primer | |
| SEQ ID NO: | Sequence (5′→3′) |

| | |
|---|---|
| 1 | GAAACTGTAAAAATAGTTCGTATAGAA . . . . . CAACAGATCAAGCCCCCTCCTGCCAAA |
| 2 | TTTGGCAGGAGGGGGCTTGATCTGTTG . . . . . TTCTATACGAACTATTTTTACAGTTTC |
| 3 | GCCAGCCCTCCGTTTCCTCTTATCGCTGAGCAGGAAATGCAGCTAGAG |
| 4 | CTCTAGCTGCATTTCCTGCTCAGCGATAAGAGGAAACGGAGGGCTGGC |
| 5 | GAAATGTCACTTTATCACCAGCCAGCAAAT . . . . . GATACTGAACCTCAGTGGG |
| 6 | GGATGGCACCCACTGAGGTTCAGTATC . . . . . ATTTGCTGGCTGGTGATAAAG |
| 7 | CAGCCAGCAAAT . . . . . GATACTGAACCTCAGTGGGTGCC |
| 8 | CACTGAGGTTCAGTATC . . . . . ATTTGCTGGCTGGTGATAAAGTGAC |
| 9 | GTCCTCACTATGCTGTGAACATATTAGACG . . . . . GTATCCGCTCATGAGACAATAAC |
| 10 | CAGGGTTATTGTCTCATGAGCGGATAC . . . . . CGTCTAATATGTTCACAGCATAGTG |
| 11 | GTGAACATATTAGACG . . . . . GTATCCGCTCATGAGACAATAACCCTG |
| 12 | GTCTCATGAGCGGATAC . . . . . CGTCTAATATGTTCACAGCATAGTGAGG |
| 13 | CTTTATCACCAGCCAGCAAAT . . . . . GATACTGAACCTCAGTGGGTG |
| 14 | CACCCACTGAGGTTCAGTATC . . . . . ATTTGCTGGCTGGTGATAAAG |
| 15 | GCTGTGAACATATTA . . . . . CCTCTTATCGCCAATGTAC |
| 16 | CATTGGCGATAAGAGG . . . . . TAATATGTTCACAGCATAGTGAGG |
| 17 | GCTTAGGCTCATTAACAAACTG . . . . . CCGGTCGCCACCATGG |
| 18 | GCAAAGGGAAGCCATG . . . . . CCTATCATCTTGATCGGTCC |
| 19 | GCCAAAGAAACTGTAATC . . . . . GAAGCCATGAAGCAAAC |
| 20 | CCAGGAGGATATCTCACTGGGTTTACAGCTTGTACAAAACAG |
| 21 | GATAGACCTAATTTTCGCCAAGGGGATTATGG |
| 22 | GCAAATACAGACCACTTCTGCAATGGAGCAGAAGCTGATCAGCGAGGAGGACCTGAATGAGAATAGCACTGTTTTGCC |
| 23 | CGACCGAAATCGAAATGCAGGGTTTAAACGATTCAAAGTTAGAAGCTGTG |
| 24 | CTGATAGACCTAATTTTCGC |
| 25 | GCCTCCAGGAGCGACTGGAAGTG |
| 26 | GAGCTGGTTTAGTGAACCGTCAGA |
| 27 | CTTGCCGTAGGTGGCATCG |
| 28 | AGGTCCTCCTCGCTGATC |

*All primers were synthesized and desalted by Gibco-BRL and used without any further purification. The dotted gaps in the primer sequences indicate positions of the deletions. The bold-faced type in the sequences for Primer SEQ ID NOs: 20 and 21 indicate the nucleotides for the point mutations. The bold-faced type in the sequences for Primer SEQ ID NOs: 22 and 23 indicate the sequences which were used for insertion and substitution, respectively.

Surprisingly, the above method was discovered to work just as well with one primer instead of two primers. For example, similar results were obtained for generating a 555-bp deletion using Primer SEQ ID NOs: 13 and 14 (Part A of table 1) versus using single Primer SEQ ID NO: 13 alone (Part B of Table 1). The primer pair gave more colonies, 1430 colonies compared with 770, but the efficiency of generating the deletion was better using the single primer (50%) as opposed to the 25% obtained with the primer pair. The lower efficiency with the primer pair might be due to the fact that during the mutagenic extension/polymerization reaction with the two complementary primers, free primer concentration was reduced because of more favorable primer-dimer formation compared to primer-template annealing. Thus, a single primer could yield more efficient polymerization than two primers.

The single primer method was also used to successfully generate a number of point mutations, deletions, insertions, and substitutions (Parts B-E of Table 1). FIG. 2B shows a single primer for making a 555 bp deletion to the GuK domain of target DNA encoding Pals1.

We also tested whether the single primer method would work well using another Escherichia coli strain such as DH5 (an mcrBC⁺ strain from Life Technologies, Rockville, Md.). The DpnI-digested DNA from an extension/polymerization reaction with just one mutagenic primer was used for transformation of DH5 cells made competent using calcium chloride (transformation efficiency 10⁷ cfu/μg) . To 50 μL competent cells, was added 2 μL of the amplified product treated with DpnI. About three times fewer clones were obtained than were obtained using EPICUREAN COLI XL-1 Blue supercompetent cells; however, the efficiency was the same. This implies that the one primer method was not restricted to one particular Escherichia coli strain. The results demonstrate that the single-step single-primer mutagenic method is useful for a variety of applications, including making large deletions. Cost of mutagenizing DNA sequences is also reduced because only one mutagenic primer need be synthesized.

The novel method of the present invention has been performed in many different reactions using a variety of cloned DNAs in plasmids and a variety of different mutagenic primers.

### EXAMPLE 2

In this example, the method of the present invention was used to insert the 30 bp Myc-tag DNA sequence in Primer SEQ ID NO: 22 (Table 2) into the Crumbs3 protein cDNA sequence (SEQ ID NO: 31; GenBank Accession No. AY103469) which had been cloned into a pCT plasmid. Primer SEQ ID NO: 22 consisted of the 30 bp Myc-tag flanked at the 5′ end by a 22 nucleotide sequence complementary to the sequence upstream of and adjacent to the insertion site and a 23 nucleotide sequence complementary to the sequence downstream of and adjacent to the insertion site.

The insertion reaction was performed as follows. A reaction mixture was prepared containing 2.5 μL of 10×PfuTURBO) reaction buffer (Stratagene), 1 μL of a 5 μM solution of Primer SEQ ID NO: 22, 0.5 μL of a 10 mM mixture of dNTP, 0.5 μL of PfuTURBO DNA polymerase (2.5 U/μL) (Stratagene), 1 μL of plasmid DNA prepared using the QIAPREP Spin miniprep kit, and water to bring the reaction mixture up to a final volume of 25 μL.

The extension polymerization conditions were 94° C. for 3 minutes, followed by 18 cycles at 94° C. for 15 seconds, 60° C. for 1 minute, and 68° C. for 14 minutes. In the last cycle, the extension polymerization was carried out at 68° C. for 5 min.

Afterwards, the amplified product from the extension/polymerization reaction was treated with 1 μL of DpnI endonuclease for 2 hours at 37° C. and 2 μL of the DpnI-digested DNA was transformed into 50 μL EPICUREAN COLI XL1 Blue supercompetent cells.

Following overnight growth of the transformants, individual colonies were picked with sterile pipette tips and smeared into 20 μL of PCR reaction mixture containing: 1×Advantage 2 reaction buffer (Clontech), 200 μM of each dNTP, 200 nM Primer SEQ ID NOs: 26 and 28 (Table 2), and 10 units of Advantage 2 DNA polymerase (Clontech). After initial heating step for 3 minutes at 95° C. to lyse the bacterial cells, PCR was carried out for 22 cycles at 94° C. for 7 seconds, 60° C. for 15 seconds, and 68° C. for 50 seconds.

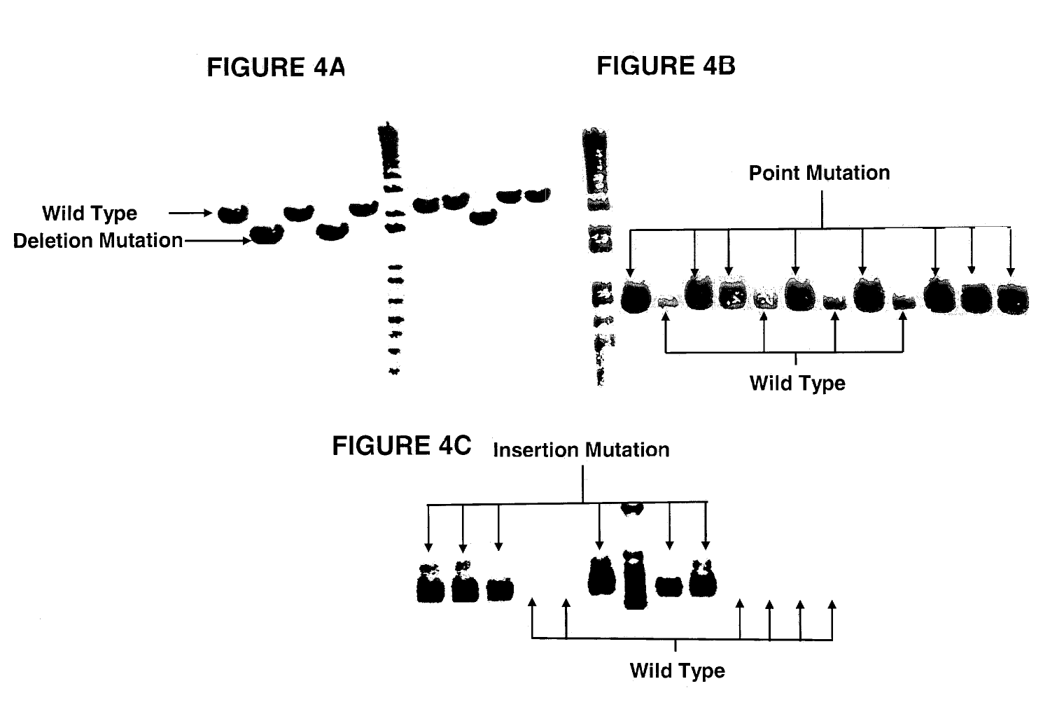

Afterwards, the PCR amplicons were electrophoresed on an 1% agarose gel and stained with ethidium bromide. The results are shown in FIG. 4C which shows that the Myc-tag had been inserted into the Crumb3 protein. The ratio between wild type and mutant colonies was used to determine the mutagenesis efficiency calculations shown in Part D of Table 1, which in this case was about 50%. The mutations were verified by DNA sequencing.

### EXAMPLE 3

In this example, the method of the present invention was used to substitute a 9 nucleotide DNA containing the 8 nucleotide PmeI restriction endonuclease sequence in Primer SEQ ID NO: 23 (Table 2) for the 174 nucleotide L27N functional domain of the Pal2 protein (SEQ ID NO: 30; GenBank Accession No. AF199009) which had been cloned into a pEYFP-N1 plasmid (Clontech) . Primer SEQ ID NO: 23 (Table 2) consisted of the 9 nucleotide DNA sequence containing the PmeI recognition sequence flanked at the 5′ end by a 20 nucleotide sequence complementary to the sequence upstream of and adjacent to the substitution site and a 21 nucleotide sequence complementary to the sequence downstream of and adjacent to the substitution site.

The substitution reaction was performed as follows. A reaction mixture was prepared containing 2.5 μL of 10×PfuTURBO) reaction buffer (Stratagene), 1 μL of a 5 μM solution of Primer SEQ ID NO: 23, 0.5 μL of a 10 mM mixture of dNTP, 0.5 μL of PfuTURBO DNA polymerase (2.5 U/μL) (Stratagene), 1 μL of plasmid DNA prepared using the QIAPREP Spin miniprep kit, and water to bring the reaction mixture up to a final volume of 25 μL.

The extension polymerization conditions were 95° C. for 1 minute, followed by 18 cycles at 94° C. for 1 minute, 52° C. for 1 minute, and 68° C. for 13 minutes. In the last cycle, the extension polymerization was carried out at 68° C. for 5 min.

Afterwards, the amplified product from the extension/polymerization reaction was treated with 1 μL of DpnI endonuclease for 2 hours at 37° C. and 2 μL of the DpnI-digested DNA was transformed into 50 μL EPICUREAN COLI XL1 Blue super competent cells.

Following overnight growth of the transformants, individual colonies were picked with sterile pipette tips and analyzed by PCR. The results are shown in Part E of Table 1 which shows that the 174 bp L27N functional domain of Pals2 was replaced by the 9 nucleotide DNA sequence containing the PmeI recognition sequence with an efficiency of about 50%. The mutation was verified by DNA sequencing.

### EXAMPLE 4

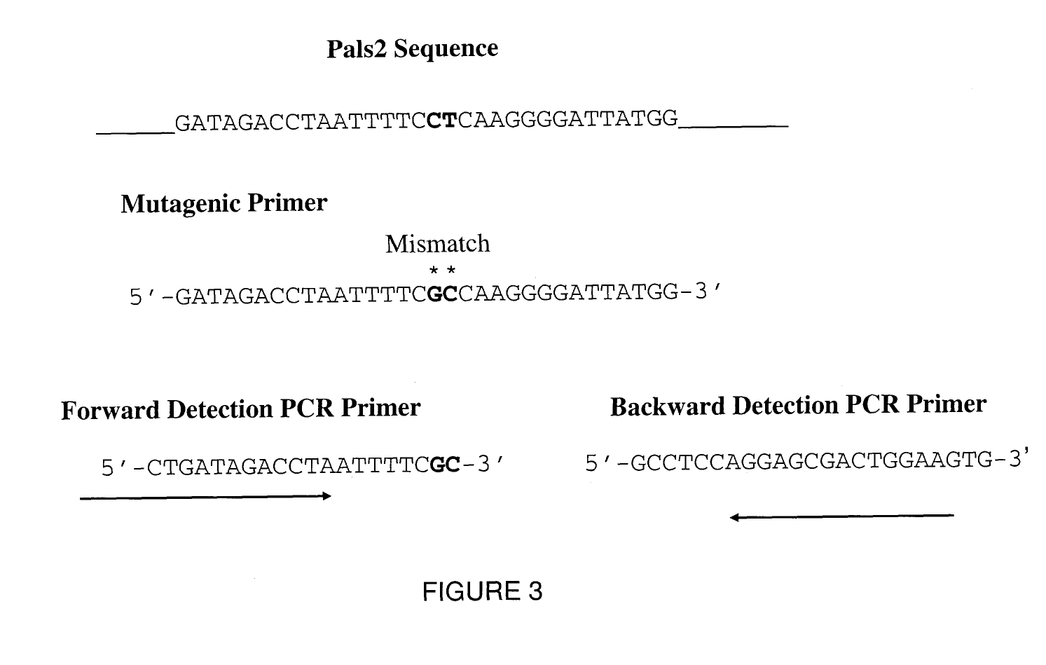

In this example, Primer SEQ ID NO: 21 (Table 2) was used to introduce a point mutation L25A (leucine to alanine substitution in codon 25) in Pals2 cDNA sequence cloned into pEYFP-N1 using the present method as schematically shown in FIG. 3. The point mutation exchanged the CT with a GC.

The point mutation reaction was performed as follows. A reaction mixture was prepared containing 2.5 μL of 10×PfuTURBO reaction buffer (Stratagene), 1 μL of a 5 μM solution of Primer SEQ ID NO: 21, 0.5 μL of a 10 mM mixture of dNTP, 0.5 μL of PfuTURBO DNA polymerase (2.5 U/μL) (Stratagene), 1 μL of plasmid DNA prepared using the QIAPREP Spin miniprep kit, and water to bring the reaction mixture up to a final volume of 25 μL.

The extension polymerization conditions were 94° C. for 3 minutes, followed by 18 cycles at 94° C. for 15 seconds, 35° C. for 1 minute, and 68° C. for 14 minutes. In the last cycle, the extension polymerization was carried out at 68° C. for 5 min.

Afterwards, the amplified product from the extension/polymerization reaction was treated with 1 μL of DpnI endonuclease for 2 hours at 37° C. and 2 μL of the DpnI-digested DNA was transformed into 50 μL EPICUREAN COLI XL1 Blue supercompetent cells.

Following overnight growth of transformants, were analyzed by PCR using Forward PCR detection Primer SEQ ID NO: 24 (Table 2) which contained the GC at the 3′ end and a downstream PCR primer, Primer SEQ ID NO: 25 (Table 2). Individual colonies were picked with sterile pipette tips and smeared into 20 μL of PCR reaction mixture containing: 1×Advantage 2 reaction buffer (Clontech), 200 μM of each dNTP, 200 nM primers (Table 2, Primer SEQ ID NOs: 24 and 25), and 10 units of Advantage 2 DNA polymerase (Clontech). After initial heating step for 3 minutes at 95° C. to lyse bacterial cells, PCR was carried out for 22 cycles at 94° C. for 7 seconds, 60° C. for 15 seconds, and 68° C. for 50 seconds.

Afterwards, the PCR amplicons were electrophoresed on an 1% agarose gel and stained with ethidium bromide. The results are shown in FIG. 4B which shows that the mutant sequence was amplified with much higher efficiency than the non-mutated DNA. The CT substitution was verified by DNA sequencing using plasmids isolated from 2 of the mutant clones. The ratio between wild type and mutant colonies was used to determine the mutagenesis efficiency calculations shown in Part C of Table 1, which in this case was about 66%.

### EXAMPLE 5

In this example, a deletion of a 555 bp DNA sequence corresponding to the GuK functional domain of Pals1 was made using Primer SEQ ID NO: 13 (Table 2).

The deletion reaction is performed as follows. A reaction mixture was prepared containing 2.5 μL of 10×PfuTURBO reaction buffer (Stratagene), 1 μL of a 5 μM solution of Primer SEQ ID NO: 5, 0.5 μL of a 10 mM mixture of dNTP, 0.5 μL of PfuTURBO DNA polymerase (2.5 U/μL) (Stratagene), 1 μL of plasmid DNA prepared using the QIAPREP Spin miniprep kit, and water to bring the reaction mixture up to a final volume of 25 μL.

The extension polymerization conditions were 94° C. for 3 minutes, followed by 18 cycles at 94° C. for 15 seconds, 51° C. for 1 minute, and 68° C. for 14 minutes. In the last cycle, the extension polymerization was carried out at 68° C. for 5 min.

Afterwards, the amplified product from the extension/polymerization reaction was treated with 1 μL of DpnI endonuclease for 2 hours at 37° C. and 2 μL of the DpnI-digested DNA was transformed into 50 μL EPICUREAN COLI XL1 Blue supercompetent cells.

Following overnight growth of transformants, individual colonies were picked with sterile pipette tips and smeared into 20 μL of PCR reaction mixture containing: 1×Advantage 2 reaction buffer (Clontech), 200 μM of each dNTP, 200 nM primers complementary to pEYFP-N1 vector flanking the Pals1 cDNA insert (Table 2, Primers SEQ ID NOs: 26 and 27), and 10 units of Advantage 2 DNA polymerase (Clontech). After an initial heating step for 3 minutes at 95° C. to lyse bacterial cells, PCR was carried out for 22 cycles at 94° C. for 7 seconds, 62° C. for 15 seconds, and 68° C. 80 seconds.

Afterwards, the PCR amplicons were electrophoresed on an 1% agarose gel and stained with ethidium bromide. FIG. 4A shows a typical example of colony screening PCR amplicons to detect DNA in which a 555 bp deletion had been made in GuK functional domain. In this Figure, the deletion was made using a primer pair consisting of Primer SEQ ID NOs: 7 and 8. The mutations were verified by DNA sequencing.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

We claim:

1. A method of introducing a mutation into a DNA sequence of interest, comprising:
(a) annealing to a target DNA comprising the DNA sequence of interest a mutagenic primer DNA or DNA analog comprising a 5′ end and a 3′ end, each end comprising a DNA sequence complementary to the DNA sequence of interest in the target DNA and wherein the mutagenic primer further includes at least one mutation located between the complementary sequences comprising 5′ and the 3′ ends of the mutagenic primer;
(b) synthesizing by means of an extension/polymerization reaction one or more mutated copies of the target DNA comprising the mutagenic primer complementary to the DNA sequence of interest linked to a DNA sequence complementary to the target DNA;
(c) providing a selection means which selectively removes or degrades the target DNA; and
(d) propagating the one or more mutated copies in host cells to produce the DNA sequence of interest with the mutation therein.

2. The method of claim 1 wherein the target DNA is single-stranded.

3. The method of claim 1 wherein the target DNA is double-stranded and the mutagenic primer is complementary to only one of the strands.

4. The method of claim 1 wherein the target DNA is methylated and the selection means is a restriction endonuclease which digests the methylated target DNA.

5. The method of claim 4 wherein the restriction endonuclease is selected from the group consisting of DpnI, MboI, NanII, NmuDI, and NmuEI.

6. The method of claim 1 wherein the target DNA is methylated and the selection means is a bacterial host with the ability to digest methylated DNA.

7. The method of claim 6 wherein the bacterial host is an Escherichia coli strain which has a genotype selected from the group consisting of mcr⁺, mrr⁺, and combinations thereof.

8. The method of claim 6 wherein the bacterial host is a strain of Escherichia coli which has an mcrBC⁺ genotype.

9. The method of claim 4 or 6 wherein the target DNA is methylated in vitro.

10. The method of claim 4 or 6 wherein the target DNA is methylated in vivo.

11. The method of claim 1 wherein the extension/polymerization reaction is catalyzed by a DNA polymerase which is substantially free of 5′ exonuclease activity and strand displacement activity.

12. The method of claim 1 wherein the extension/polymerization reaction is catalyzed by a DNA polymerase selected from the group consisting of Pfu DNA polymerase, pfx DNA polymerase, Taq DNA polymerase, and modified variants thereof.

13. The method of claim 1 wherein the extension/polymerization reaction is repeated for 35 cycles or less.

14. The method of claim 1 wherein the host cells are procaryotic cells or eucaryotic cells.

15. The method of claim 1 wherein the target DNA is circular.

16. The method of claim 1 wherein the mutation is selected from the group consisting of deletion, insertion, substitution, point mutation, and combinations thereof.

17. A method for introducing at least one mutation into a DNA sequence of interest, comprising:
(a) providing a double-stranded circular target DNA comprising the DNA sequence of interest and wherein one strand is a template strand and the other strand is a non-template strand;
(b) providing a mutagenic primer DNA or DNA analog which includes a 5′ end and a 3′ end, each end comprising a DNA sequence complementary to the DNA sequence of interest in the template strand and wherein the mutagenic primer further includes at least one mutation located between complementary sequences at the 5′ and the 3′ ends;
(c) denaturing the double-stranded circular target DNA to separate the template strand from the non-template strand;
(d) annealing the mutagenic primer to the template strand to form an annealed product;
(e) subjecting the annealed product to an extension/polymerization reaction using a DNA polymerase to produce an amplified product containing one or more of mutated DNA molecules comprising the mutagenic primer and a DNA sequence complementary to the template strand;
(f) using a selection means to remove or degrade the target DNA; and
(g) propagating the one or more mutated DNA molecules in host cells to produce the DNA sequence of interest with the at least one mutation.

18. The method of claim 17 wherein the target DNA is methylated and the selection means is a restriction endonuclease which digests the methylated target DNA.

19. The method of claim 17 wherein the restriction endonuclease is selected from the group consisting of DpnI, MboI, NanII, NmuDI, and NmuEI.

20. The method of claim 17 wherein the target DNA is methylated and the selection means is a bacterial host with the ability to digest methylated DNA.

21. The method of claim 20 wherein the bacterial host is an Escherichia coli strain which has a genotype selected from the group consisting of mcr⁺, mrr⁺, and combinations thereof.

22. The method of claim 20 wherein the bacterial host is a strain of Escherichia coli which has an mcrBC⁺ genotype.

23. The method of claim 18 or 20 wherein the target DNA is methylated in vitro.

24. The method of claim 18 or 20 wherein the target DNA is methylated in vivo.

25. The method of claim 17 wherein the extension/polymerization reaction is catalyzed by a DNA polymerase which is substantially free of 5′ exonuclease activity and strand displacement activity.

26. The method of claim 17 wherein the extension/polymerization reaction is catalyzed by a DNA polymerase selected from the group consisting of Pfu DNA polymerase, pfx DNA polymerase, Taq DNA polymerase, and modified variants thereof.

27. The method of claim 17 wherein the extension/polymerization reaction is repeated for 35 cycles or less.

28. The method of claim 17 wherein the host cells are procaryotic cells or eucaryotic cells.

29. The method of claim 17 wherein the mutation is selected from the group consisting of deletion, insertion, substitution, point mutation, and combinations thereof.

30. A method for producing a library of mutations in a DNA sequence of interest, comprising:
(a) annealing to a target DNA comprising the DNA sequence of interest a plurality of mutagenic primer DNAs or DNA analogs, each comprising a 5′ end and a 3′ end, each end comprising a DNA sequence complementary to the DNA sequence of interest in the target DNA, and each mutagenic primer including at least one mutation located between the complementary sequences comprising 5′ and the 3′ ends of the mutagenic primer;
(b) synthesizing by means of an extension/polymerization reaction a plurality of mutated copies of the target DNA, each copy comprising one of the mutagenic primers complementary to the DNA sequence of interest linked to a DNA sequence complementary to the target DNA;
(c) providing a selection means which selectively removes or degrades the target DNA; and
(d) propagating the plurality of mutated copies of the target DNA in host cells to produce the library of mutations in the DNA sequence of interest.

31. The method of claim 30 wherein the target DNA is single-stranded.

32. The method of claim 30 wherein the target DNA is double-stranded and the mutagenic primers are each complementary to the same strand.

33. The method of claim 30 wherein the target DNA is methylated and the selection means is a restriction endonuclease which digests the methylated target DNA.

34. The method of claim 33 wherein the restriction endonuclease is selected from the group consisting of DpnI, MboI, NanII, NmuDI, and NmuEI.

35. The method of claim 30 wherein the target DNA is methylated and the selection means is a bacterial host with the ability to digest methylated DNA.

36. The method of claim 35 wherein the bacterial host is an Escherichia coli strain which has a genotype selected from the group consisting of mcr⁺, mrr⁺, and combinations thereof.

37. The method of claim 35 wherein the bacterial host is a strain of Escherichia coli which has an mcrBC⁺ genotype.

38. The method of claim 33 or 35 wherein the target DNA is methylated in vitro.

39. The method of claim 33 or 35 wherein the target DNA is methylated in vivo.

40. The method of claim 30 wherein the extension/polymerization reaction is catalyzed by a DNA polymerase which is substantially free of 5′ exonuclease activity and strand displacement activity.

41. The method of claim 30 wherein the extension/polymerization reaction is catalyzed by a DNA polymerase selected from the group consisting of Pfu DNA polymerase, pfx DNA polymerase, Taq DNA polymerase, and modified variants thereof.

42. The method of claim 30 wherein the extension/polymerization reaction is repeated for 35 cycles or less.

43. The method of claim 30 wherein the host cells are procaryotic cells or eucaryotic cells.

44. The method of claim 30 wherein the target DNA is circular.

45. The method of claim 30 wherein the mutation is selected from the group consisting of deletion, insertion, substitution, point mutation, and combinations thereof.

46. A kit for introducing a specific mutation into a DNA molecule, comprising:
(a) a DNA polymerase substantially free of 5′ exonuclease activity and strand displacement activity;
(b) an endonuclease which degrades DNA without the specific mutation;
(c) a control DNA molecule; and
(d) a control mutagenic primer, wherein the control mutagenic primer includes a 5′ end and a 3′ end, each end comprising a DNA sequence complementary to the control DNA molecule and at least one mutation site located between the complementary 5′ and the 3′ ends.

47. The kit of claim 46 wherein the endonuclease is selected from the group consisting of DpnI, NanII, NmuDI, and NmuEI.

48. The kit of claim 46 wherein the DNA polymerase is selected from the group consisting of Pfu DNA polymerase, pfx DNA polymerase, Taq DNA polymerase, and modified variants thereof.

49. The kit of claim 46 further including competent cells.

50. The kit of claim 49 wherein the competent cells are bacterial cells.

51. The kit of claim 50 wherein the bacterial cells are capable of degrading the DNA without the specific mutation.

52. The kit of claim 50 further including concentrated reaction buffers.

53. The kit of claim 50 further including a methylase.
